(19) United States
(12) Patent Application Publication
(10) Pub. No.: US 2022/0117474 A1
(43) Pub. Date: Apr. 21, 2022

(54) IMAGE PROCESSING APPARATUS, ENDOSCOPE SYSTEM, AND OPERATION METHOD OF IMAGE PROCESSING APPARATUS

(71) Applicant: FUJIFILM Corporation, Tokyo (JP)

(72) Inventor: Hiroki WATANABE, Kanagawa (JP)

(73) Assignee: FUJIFILM Corporation, Tokyo (JP)

(21) Appl. No.: 17/565,040

(22) Filed: Dec. 29, 2021

Related U.S. Application Data
(63) Continuation of application No. PCT/JP2020/025695, filed on Jun. 30, 2020.

(30) Foreign Application Priority Data
Jul. 8, 2019 (JP) 2019-127010

(51) Int. Cl. A61B 1/05; G06T 7/00; A61B 1/00

(52) U.S. Cl. CPC A61B 1/05; G06T 7/0012; G06T 2207/30101; G06T 2207/10068; A61B 1/00009

(57) ABSTRACT

An image acquisition unit acquires a medical image which is obtained by picking up an image of an observation target illuminated with illumination light including short-wavelength narrowband light, the observation target being magnified at a first magnification ratio or more and less than a second magnification ratio that is more than the first magnification ratio. A disease-related processing unit performs processing related to the disease on the basis of the medical image.

### CROSS-REFERENCE TO RELATED APPLICATIONS

This application is a Continuation of PCT International Application No. PCT/JP2020/025695 filed on 30 Jun. 2020, which claims priority under 35 U.S.C § 119(a) to Japanese Patent Application No. 2019-127010 filed on 8 Jul. 2019. The above application is hereby expressly incorporated by reference, in its entirety, into the present application.

### BACKGROUND OF THE INVENTION

### 1. Field of the Invention

The present invention relates to an image processing apparatus that performs processing related to a disease, an endoscope system, and an operation method of an image processing apparatus.

### 2. Description of the Related Art

In the medical field, a medical image has been widely used for diagnosis. For example, as an apparatus using a medical image, there is an endoscope system comprising a light source apparatus, an endoscope, and a processor apparatus. In the endoscope system, an observation target is irradiated with illumination light and an image of the observation target illuminated with the illumination light is picked up, so that an endoscopic image as the medical image is acquired. The endoscopic image is displayed on a monitor and used for diagnosis.

Further, in recent years, with processing performed on the basis of the endoscopic image, information that is used to support diagnosis, for example, determination regarding a disease, such as a lesion, has been also provided to a user. For example, in JP2016-154810A, a feature amount is calculated from an endoscopic image, and classification (for example, non-tumor, tumor, cancer, and SSA/P) corresponding to pathological diagnosis is performed on the basis of the feature amount.

### SUMMARY OF THE INVENTION

As a method of determining a disease, for example, there is a method of determining a disease (for example, ulcerative colitis) on the basis of a property of a microvessel or a bleeding area. In this case, since imaging with low magnification ratio of various elements of a living body, such as a microvessel or a bleeding area, makes the blood vessel or bleeding area imaged very small, it is difficult to extract the blood vessel or bleeding area in a state in which the actual shape of the blood vessel or bleeding area is maintained, upon the extraction. On the other hand, in a case where the elements of the living body, such as the microvessel or bleeding area are imaged at an excessively high magnification ratio, not entire shape information of the elements of the living body, as an observation target, but only local shape information of the elements of the living body may be obtained. Therefore, it has been required that a medical image in which the observation target is imaged at an appropriate magnification ratio is used, to enable extraction in a state in which the shape of the microvessel or bleeding area is maintained and improvement of accuracy of processing related to the disease.

JP2016-154810A describes that an image in which a tissue is magnified, for example, a 380 times magnified image is used as an endoscopic image. However, there is no description or suggestion of an appropriate magnification ratio range for diagnostic imaging.

An object of the present invention is to provide an image processing apparatus, an endoscope system, and an operation method of an image processing apparatus in which a medical image in which an observation target is imaged at an appropriate magnification ratio is used to enable extraction in a state in which a shape of a microvessel or a bleeding area is maintained and improvement of accuracy of processing related to a disease.

According to the present invention, there is provided an image processing apparatus comprising a processor, in which the processor acquires a medical image which is obtained by picking up an image of an observation target illuminated with illumination light including short-wavelength narrowband light, the observation target being magnified at a first magnification ratio or more and less than a second magnification ratio that is more than the first magnification ratio, and performs processing related to a disease on the basis of the medical image.

It is preferable that magnification of the observation target is performed at the first magnification ratio, to make a thickness of a blood vessel that is included in the observation target magnified to one or more pixels. The first magnification ratio is preferably five times or more. The second magnification ratio is preferably 230 times or less.

The illumination light is preferably violet light of which a central wavelength or a peak wavelength includes a wavelength of 410 nm, as the short-wavelength narrowband light. It is preferable that the illumination light is blue narrowband light and green narrowband light, as the short-wavelength narrowband light, and the medical image is obtained by picking up an image of the observation target that is alternately illuminated with the blue narrowband light and the green narrowband light. The illumination light is preferably pseudo white light including the short-wavelength narrowband light and fluorescence obtained by irradiating a phosphor with excitation light. The illumination light preferably includes violet light as the short-wavelength narrowband light and blue light, green light, or red light.

It is preferable that the processor performs at least one of calculating an index value for a stage of ulcerative colitis, determining the stage of the ulcerative colitis, or determining pathological remission or pathological non-remission of the ulcerative colitis, on the basis of at least one of superficial vascular congestion, intramucosal bleeding, or extramucosal bleeding that is obtained from the medical image.

According to the present invention, there is provided an endoscope system comprising: a light source unit that emits illumination light including short-wavelength narrowband light; and a processor, in which the processor acquires a medical image which is obtained by picking up an image of an observation target illuminated with the illumination light, the observation target being magnified at a first magnification ratio or more and less than a second magnification ratio that is more than the first magnification ratio, and performs processing related to a disease on the basis of the medical image.

According to the present invention, there is provided an operation method of an image processing apparatus in which a processor executes: a step of acquiring a medical image which is obtained by picking up an image of an observation target illuminated with illumination light including short-wavelength narrowband light, the observation target being magnified at a first magnification ratio or more and less than a second magnification ratio that is more than the first magnification ratio; and a step of performing processing related to a disease on the basis of the medical image.

According to the present invention, a medical image in which an observation target is imaged at an appropriate magnification ratio is used to enable extraction in a state in which a shape of a microvessel or a bleeding area is maintained and improvement of accuracy of processing related to a disease.

### BRIEF DESCRIPTION OF THE DRAWINGS

FIG. 1 is an appearance diagram of an endoscope system.

FIG. 2 is a block diagram showing a function of an endoscope system according to a first embodiment.

FIG. 3 is a graph showing spectra of violet light V, blue light B, green light G, and red light R.

FIG. 4 is a graph showing a spectrum of special light according to the first embodiment.

FIG. 5 is a graph showing a spectrum of special light including only violet light V.

FIG. 6 is a diagram illustrating a magnification ratio display section that is displayed in a case where a magnification ratio is changed stepwise and a magnification ratio display section that is displayed in a case where a magnification ratio is continuously changed.

FIGS. 7A to 7E are diagrams illustrating a pattern of a vascular structure that varies depending on severity of ulcerative colitis.

FIG. 8 is a cross-sectional view showing a cross-section of the large intestine.

FIG. 9 is a block diagram showing a function of a disease-related processing unit.

FIG. 10 is an image diagram of a monitor that displays information regarding determination.

FIGS. 11A and 11B are diagrams illustrating blood vessel extraction performed on a special light image in a case where a thickness of a blood vessel is smaller than one pixel.

FIGS. 12A and 12B are diagrams illustrating blood vessel extraction performed on a special light image in a case where the thickness of the blood vessel is one or more pixels.

FIG. 13 is a graph showing a relationship between an index value and a pathological score in a case where a second magnification ratio is 40 times.

FIG. 14 is a graph showing a relationship between an index value and a pathological score in a case where the second magnification ratio is 135 times.

FIG. 15 is an image diagram showing a special light image in which a vascular density differs depending on a position.

FIG. 16 is a flowchart showing a series of flow of a disease-related processing mode.

FIG. 17 is a block diagram showing a function of an endoscope system according to a second embodiment.

FIG. 18 is a plan view of a rotation filter.

FIG. 19 is a block diagram showing a function of an endoscope system of a third embodiment.

FIG. 20 is a graph showing a spectrum of normal light according to the third embodiment.

FIG. 21 is a graph showing a spectrum of special light according to the third embodiment.

FIG. 22 is a block diagram showing a diagnosis support apparatus.

FIG. 23 is a block diagram showing a medical service support apparatus.

### DESCRIPTION OF THE PREFERRED EMBODIMENTS

### First Embodiment

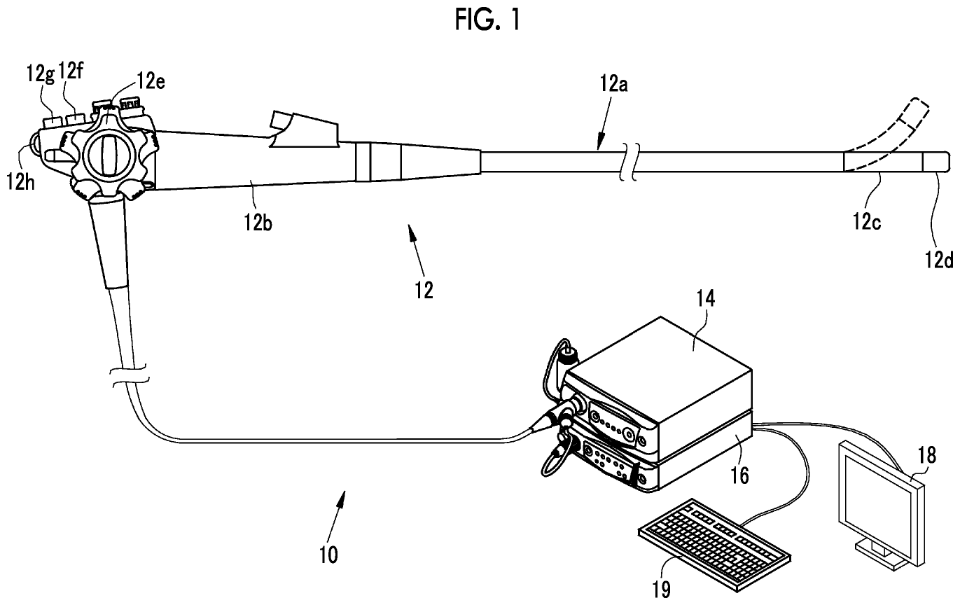

In FIG. 1, an endoscope system 10 includes an endoscope 12, a light source apparatus 14, a processor apparatus 16, a monitor 18, and a user interface 19. The endoscope 12 is optically connected to the light source apparatus 14 and electrically connected to the processor apparatus 16. The endoscope 12 includes an insertion part 12a that is inserted into a body as an observation target, an operation part 12b that is provided in a proximal end part of the insertion part 12a, and a bendable part 12c and a distal end part 12d that are provided on a distal end side of the insertion part 12a. In a case where an angle knob 12e of the operation part 12b is operated, the bendable part 12c is operated to be bent. In a case where the bendable part 12c is operated to be bent, the distal end part 12d is directed in a desired direction.

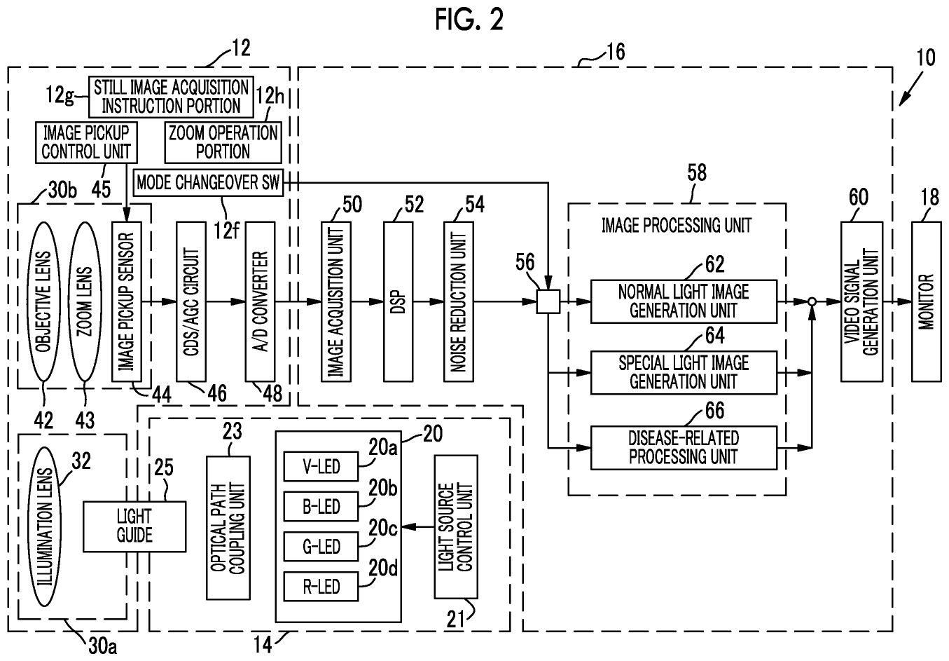

Further, the operation part 12b is provided with a mode changeover SW (mode changeover switch) 12f that is used for mode switching operation, a still image acquisition instruction portion 12g that is used for an acquisition instruction of a still image of the observation target, and a zoom operation portion 12h that is used for operation of a zoom lens 43 (see FIG. 2), in addition to the angle knob 12e.

The endoscope system 10 has three modes of a normal light mode, a special light mode, and a disease-related processing mode. In the normal light mode, an observation target is illuminated with normal light and an image thereof is picked up, so that a normal light image having natural color is displayed on a monitor 18. In the special light mode, the observation target is illuminated with special light having a wavelength range different from the normal light and an image thereof is picked up, so that a special light image in which a specific structure is enhanced is displayed on the monitor 18. In the disease-related processing mode, pathological remission or pathological non-remission of ulcerative colitis, which is one of the diseases, is determined on the basis of the normal light image or the special light image. In the disease-related processing mode, an index value for a stage of ulcerative colitis may be calculated, or the stage of ulcerative colitis may be determined.

In the present embodiment, a special light image (endoscopic image) is used in the disease-related processing mode, but a normal light image may be used. As the image that is used in the disease-related processing mode, a medical image, such as a radiography image that is obtained by a radiography apparatus, a CT image that is obtained by a computed tomography (CT) apparatus, and an MRI image obtained by a magnetic resonance imaging (MRI), may be used, in addition to the special light image as the endoscopic image which is one of the medical images. Further, the processor apparatus 16 to which the endoscope 12 is connected corresponds to an image processing apparatus according to the present invention, and the processor apparatus 16 executes the disease-related processing mode, but the disease-related processing mode may be executed by another method. For example, an external image processing apparatus different from the endoscope system 10 may be provided with a function of a disease-related processing unit 66, the external image processing apparatus may execute the disease-related processing mode in response to input of the medical image, and the execution result may be displayed on an external monitor connected to the external image processing apparatus.

The processor apparatus 16 is electrically connected to the monitor 18 and the user interface 19. The monitor 18 outputs and displays an image of the observation target, information incidental to the image of the observation target, and the like. The user interface 19 has a function of receiving input operation, such as function settings. An external recording unit (not shown) that is used to record an image, image information, or the like may be connected to the processor apparatus 16. Further, the processor apparatus 16 corresponds to the image processing apparatus according to the present invention.

In FIG. 2, the light source apparatus 14 comprises a light source unit 20 and a light source control unit 21 that controls the light source unit 20. The light source unit 20 has, for example, a plurality of semiconductor light sources each of which is turned on or off, and in a case where the semiconductor light source is turned on, the light source unit 20 controls an amount of light emitted from each semiconductor light source to emit illumination light with which the observation target is illuminated. In the present embodiment, the light source unit 20 has four-color LEDs of a violet light emitting diode (V-LED) 20a, a blue light emitting diode (B-LED) 20b, a green light emitting diode (G-LED) 20c, and a red light emitting diode (R-LED) 20d.

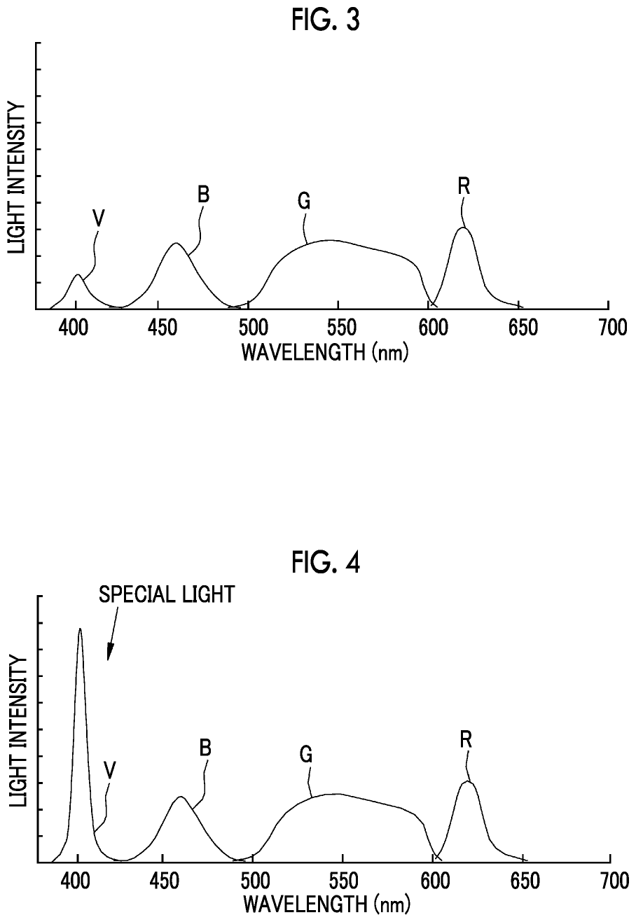

As shown in FIG. 3, the V-LED 20a generates violet light V having a central wavelength of 405±10 nm and a wavelength range of 380 to 420 nm. The B-LED 20b generates blue light B having a central wavelength of 460±10 nm and a wavelength range of 420 to 500 nm. The G-LED 20c generates green light G having a wavelength range of 480 to 600 nm. The R-LED 20d generates red light R having a central wavelength of 620 to 630 nm and a wavelength range of 600 to 650 nm. The violet light V is a short-wavelength narrowband light that is used to detect superficial vascular congestion, intramucosal bleeding, and extramucosal bleeding used in the disease-related processing mode, and a central wavelength or a peak wavelength thereof includes preferably a wavelength of 410 nm.

The light source control unit 21 controls the V-LED 20a, the B-LED 20b, the G-LED 20c, and the R-LED 20d. Further, the light source control unit 21 controls each of the LEDs 20a to 20d so that normal light of which a light intensity ratio of violet light V, blue light B, green light G, and red light R is Vc:Bc:Gc:Rc is emitted, in the normal light mode.

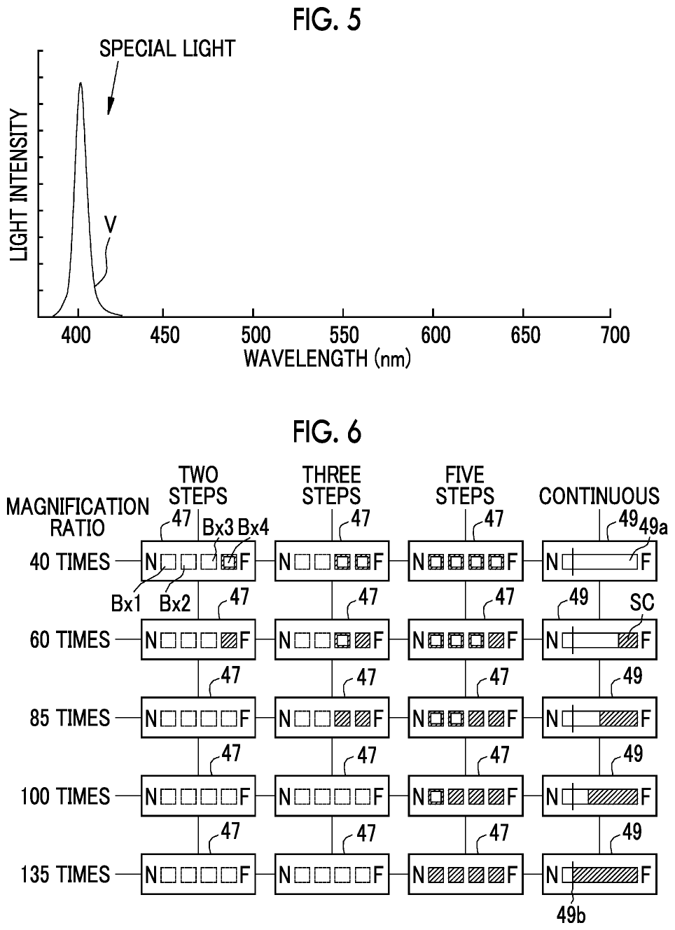

Further, the light source control unit 21 controls each of the LEDs 20a to 20d so that special light of which a light intensity ratio of violet light V as the short-wavelength narrowband light, and blue light B, green light G, and red light R is Vs:Bs:Gs:Rs is emitted, in the special light mode and the disease-related processing mode. It is preferable that a superficial blood vessel and the like are enhanced by the special light having the light intensity ratio of Vs:Bs:Gs:Rs. Therefore, in first illumination light, it is preferable that the light intensity of violet light V is larger than the light intensity of the blue light B. For example, as shown in FIG. 4, the ratio of the light intensity Vs of violet light V to the light intensity Bs of blue light B is set to “4:1”. Alternatively, as shown in FIG. 5, for special light, the light intensity ratio of violet light V, blue light B, green light G, and red light R may be set to 1:0:0:0, and only the violet light V as the short-wavelength narrowband light may be emitted.

In the present specification, the light intensity ratio includes a case where the ratio of at least one semiconductor light source is 0 (zero). Therefore, a case where any one or more of the semiconductor light sources are not turned on is included. For example, even in a case where only one of the semiconductor light sources is turned on and the other three thereof is not turned on as in a case where the light intensity ratio of violet light V, blue light B, green light G, and red light R is 1:0:0:0, light is assumed to have the light intensity ratio.

The light emitted by each of the LEDs 20a to 20e is incident on a light guide 25 via an optical path coupling unit 23 that is formed of a mirror, a lens, or the like. The light guide 25 is built in the endoscope 12 and the universal cord (cord that connects the endoscope 12 to the light source apparatus 14 and the processor apparatus 16). The light guide 25 propagates the light from the optical path coupling unit 23 to the distal end part 12d of the endoscope 12.

The distal end part 12d of the endoscope 12 is provided with an illumination optical system 30a and an image pickup optical system 30b. The illumination optical system 30a has an illumination lens 32, and the observation target is irradiated with the illumination light propagated by the light guide 25 via the illumination lens 32. The image pickup optical system 30b has an objective lens 42 and an image pickup sensor 44. The light from the observation target irradiated with the illumination light is incident on the image pickup sensor 44, via the objective lens 42 and a zoom lens 43. As a result, an image of the observation target is formed on the image pickup sensor 44. The zoom lens 43 is a lens that is used to magnify the observation target, and the zoom operation portion 12h is operated, to move the zoom lens 43 between a telephoto end and a wide-angle end.

In the present embodiment, the zoom lens 43 is used to change the magnification ratio stepwise. Here, the magnification ratio is a value that is obtained by dividing the size of an object displayed on the monitor 18 by the size of an actual object. For example, in a case where the monitor 18 is a 19-inch monitor, as shown in FIG. 6, the magnification ratio can be changed stepwise in two steps, three steps, and five steps, or the magnification ratio can be continuously changed. In order to display the magnification ratio in use on the monitor 18, a magnification ratio display section 47 that is displayed in a case where the magnification ratio is changed stepwise and a magnification ratio display section 49 that is displayed in a case where the magnification ratio is continuously changed are provided at a specific display position of the monitor 18. In the magnification ratio display section 47, the magnification ratio in use is represented by a combination of frameless display, framed display, and full display of boxes Bx1, Bx2, Bx3, and Bx4 provided between Near (N) representing a near field and Far (F) representing a far field. The size of the monitor 18 generally used in the endoscope system 10 is 19 inches to 32 inches, and the width thereof is 23.65 cm to 39.83 cm.

Specifically, in a case where the magnification ratio is set to be changed in two steps in which the magnification ratio is changed to 40 times and 60 times, the frameless display is used for the boxes Bx1, Bx2, and Bx3, the framed display is used for the box Bx4 in a case of the magnification ratio in use of 40 times, and the full display is used for the box Bx4 in a case of the magnification ratio in use of 60 times. Alternatively, in a case where the magnification ratio is set to be changed in three steps in which the magnification ratio is changed to 40 times, 60 times, and 85 times, the frameless display is used for the boxes Bx1 and Bx2, and the framed display is used for the boxes Bx3 and Bx4 in a case of the magnification ratio in use of 40 times. The framed display is used for the box Bx3, and the full display is used for the box Bx4 in a case of the magnification ratio in use of 60 times, and the full display is used for the boxes Bx3 and Bx4 in a case of the magnification ratio in use of 85 times.

Alternatively, in a case where the magnification ratio is set to be changed in five steps of 40 times, 60 times, 85 times, 100 times, and 135 times, the framed display is used for the boxes Bx1, Bx2, Bx3, and Bx4 in a case of the magnification ratio in use of 40 times. Further, the framed display is used for the boxes Bx1, Bx2, and Bx3, and the full display is used for the box Bx4 in a case of the magnification ratio in use of 60 times. Further, the framed display is used for the boxes Bx1 and Bx2, and the full display is used for the boxes Bx3 and Bx4 in a case of the magnification ratio of 85 times. Further, the framed display is used for the box Bx1, and the full display is used for the boxes Bx2, Bx3, and Bx4 in a case of the magnification ratio of 100 times. Further, the full display is used for the boxes Bx1, Bx2, Bx3, and Bx4 in a case of the magnification ratio of 135 times.

The magnification ratio display section 49 is provided with a horizontal bar 49a provided between Near (N) representing a near field and Far (F) representing a far field. Only the frame of the horizontal bar 49a is displayed until the magnification ratio becomes 40 times. In a case where the magnification ratio exceeds 40 times, the inside of the frame of the horizontal bar 49a is displayed in specific color SC. Until the magnification ratio reaches 135 times, the region of the specific color in the horizontal bar 49a gradually expands to the N side as the magnification ratio is increased. In a case where the magnification ratio reaches 135 times, the region of the specific color expands to an upper limit display bar 49b, and does not further expand to the N side.

As the image pickup sensor 44, a charge coupled device (CCD) image pickup sensor or a complementary metal-oxide semiconductor (CMOS) image pickup sensor may be used. Further, instead of the primary color image pickup sensor 44, a complementary color image pickup sensor provided with complementary color filters of cyan (C), magenta (M), yellow (Y), and green (G) may be used. In a case where the complementary color image pickup sensor is used, image signals of the four colors of CMYG are output. Therefore, the image signals of the four colors of CMYG are converted into image signals of the three colors of RGB by the complementary color-primary color conversion, so that an image signal of each color of the same RGB as that of the image pickup sensor 44 can be obtained.

The image pickup sensor 44 is driven and controlled by the image pickup control unit 45. The control by the image pickup control unit 45 differs for each mode. In the normal light mode, the image pickup control unit 45 controls the image pickup sensor 44 so as to pick up an image of the observation target illuminated with the normal light. Accordingly, a Bc image signal is output from a B pixel of the image pickup sensor 44, a Gc image signal is output from a G pixel, and an Rc image signal is output from an R pixel.

In the special light mode or the disease-related processing mode, the image pickup control unit 45 controls the image pickup sensor 44 so as to pick up an image of the observation target illuminated with the special light. Accordingly, a Bs image signal is output from the B pixel of the image pickup sensor 44, a Gs image signal is output from the G pixel, and an Rs image signal is output from the R pixel.

A correlated double sampling/automatic gain control (CDS/AGC) circuit 46 performs correlated double sampling (CDS) and automatic gain control (AGC) on an analog image signal obtained from the image pickup sensor 44. The image signal that has passed through the CDS/AGC circuit 46 is converted into a digital image signal by an analog/digital (A/D) converter 48. The digital image signal after A/D conversion is input to the processor apparatus 16.

The processor apparatus 16 comprises an image acquisition unit 50, a digital signal processor (DSP) 52, a noise reduction unit 54, an image processing switching unit 56, an image processing unit 58, and a video signal generation unit 60. The image processing unit 58 comprises a normal light image generation unit 62, a special light image generation unit 64, and a disease-related processing unit 66.

In the processor apparatus 16, programs related to various processing are stored in a program storage memory (not shown). With the programs in the program storage memory to be executed by the processor, the functions of the image acquisition unit 50, the noise reduction unit 54, the image processing switching unit 56, the image processing unit 58, and a video signal generation unit 60 are realized. Along with this, the functions of the normal light image generation unit 62, the special light image generation unit 64, and the disease-related processing unit 66 included in the image processing unit 58 are realized.

The image acquisition unit 50 acquires an image signal of an endoscopic image, which is one of medical images that are input from the endoscope 12. The acquired image signal is transmitted to the DSP 52. The DSP 52 performs various signal processing such as defect correction processing, offset processing, gain correction processing, linear matrix processing, gamma conversion processing, demosaicing processing, and YC conversion processing, on the received image signal. In the defect correction processing, a signal of a defective pixel of the image pickup sensor 44 is corrected. In the offset processing, a dark current component is removed from the image signal subjected to the defect correction processing, and an accurate zero level is set. In the gain correction processing, the image signal of each color after the offset processing is multiplied by a specific gain and the signal level of each image signal is adjusted. The image signal of each color after the gain correction processing is subjected to linear matrix processing for enhancing color reproducibility.

After that, the brightness and saturation of each image signal are adjusted by the gamma conversion processing. The image signal after the linear matrix processing is subjected to the demosaicing processing (also referred to as isotropic processing or demosaicking processing), and a signal of the missing color of each pixel is generated by interpolation. With the demosaicing processing, all the pixels have a signal of each color of RGB. The DSP 52 performs YC conversion processing on each image signal after demosaicing processing and outputs a brightness signal Y and a color difference signals Cb and Cr to the noise reduction unit 54.

The noise reduction unit 54 performs noise reduction processing using, for example, a moving average method, a median filter method, on the image signal subjected to the demosaicing processing by the DSP 52 and the like. The image signal with reduced noise is input to the image processing switching unit 56.

The image processing switching unit 56 switches a transmission destination of the image signal from the noise reduction unit 54 to any one of the normal light image generation unit 62, the special light image generation unit 64, or the disease-related processing unit 66, on the basis of the set mode. Specifically, in a case where the normal light mode is set, the image signal from the noise reduction unit 54 is input to the normal light image generation unit 62. In a case where the special light mode is set, the image signal from the noise reduction unit 54 is input to the special light image generation unit 64. In a case where the disease-related processing mode is set, the image signal from the noise reduction unit 54 is input to the disease-related processing unit 66.

The normal light image generation unit 62 performs image processing for normal light image, on the input Rc image signal, Gc image signal, and Bc image signal for one frame. The image processing for normal light image includes color conversion processing such as 3×3 matrix processing, gradation conversion processing, and three-dimensional look up table (LUT) processing, and structure enhancement processing such as color enhancement processing and spatial frequency enhancement. The Rc image signal, the Gc image signal, and the Bc image signal subjected to the image processing for normal light image are input to the video signal generation unit 60 as a normal light image.

The special light image generation unit 64 performs image processing for special light image, on the input Rs image signal, Gs image signal, and Bs image signal for one frame. The image processing for special light image includes color conversion processing such as 3×3 matrix processing, gradation conversion processing, and three-dimensional look up table (LUT) processing, and structure enhancement processing such as color enhancement processing and spatial frequency enhancement. The Rs image signal, Gs image signal, and Bs image signal subjected to image processing for special light image are input to the video signal generation unit 60 as a special light image.

The disease-related processing unit 66 performs disease-related processing on the basis of a special light image which is one of medical images. Specifically, the disease-related processing unit 66 performs at least one of calculating an index value for a stage of ulcerative colitis, determining the stage of the ulcerative colitis, or determining pathological remission or pathological non-remission of the ulcerative colitis, on the basis of superficial vascular congestion, intramucosal bleeding, and extramucosal bleeding that are obtained from the special light image. Information regarding the determination result is input to the video signal generation unit 60. Details of the disease-related processing unit 66 will be described later. In the first to third embodiments, a case where the disease-related processing unit 66 determines pathological remission or pathological non-remission of ulcerative colitis will be described.

The video signal generation unit 60 converts the normal light image, the special light image, or the information regarding the determination result output from the image processing unit 58 into a video signal that can be displayed in full color on the monitor 18. The converted video signal is input to the monitor 18. As a result, the normal light image, the special light image, or the information regarding the determination result is displayed on the monitor 18.

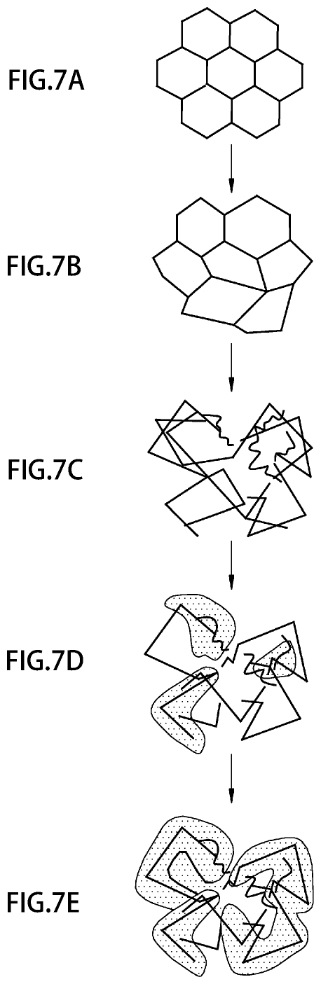

The details of the disease-related processing unit 66 will be described below. As shown in FIGS. 7A to 7E, the inventors have found out that ulcerative colitis to be determined by the disease-related processing unit 66 changes the pattern of the vascular structure as the severity worsens. In a case where ulcerative colitis is in pathological remission or ulcerative colitis does not occur, the pattern of superficial blood vessels is regular (FIG. 7A), or some disturbance occurs in the pattern regularity of the superficial blood vessels (FIG. 7B). On the other hand, in a case where ulcerative colitis is in pathological non-remission and the severity is mild, superficial blood vessels are locally congested (FIG. 7C). Further, in a case where ulcerative colitis is in pathological non-remission and the severity is moderate, intramucosal bleeding occurs (FIG. 7D). Further, in a case where ulcerative colitis is in pathological non-remission and the severity is moderate to severe, extramucosal bleeding occurs (FIG. 7E). The disease-related processing unit 66 determines pathological remission or pathological non-remission of ulcerative colitis on the basis of the special light image which is one of the medical images, by using the pattern change of the vascular structure.

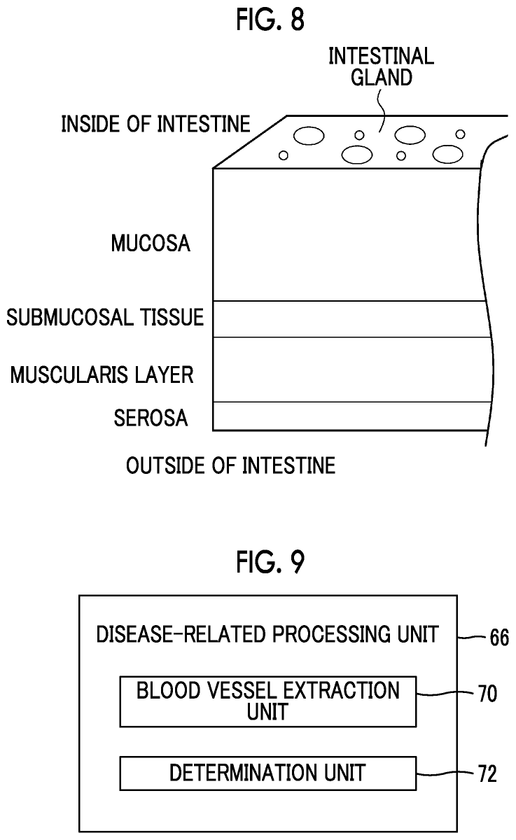

Here, the “superficial vascular congestion” refers to a state in which superficial blood vessels meander and gather, and in appearance on the image, the intestinal gland (crypt) is surrounded by many superficial blood vessels (see FIG. 8). The “intramucosal bleeding” refers to bleeding in the mucosal tissue (see FIG. 7D), and is required to be discriminated from bleeding into the intracavity. The “intramucosal bleeding” refers to bleeding not in the interior of the mucosa and in the intracavity (lumen, cavity of fold), in appearance on the image. The “extramucosal bleeding” refers to a small amount of blood into the lumen, visible blood that oozes out of the mucosa or the lumen in front of the endoscope after cleaning of the lumen, or blood in the lumen that oozes through bleeding mucosa.

The disease-related processing unit 66 performs disease-related processing on the basis of the special light image. Specifically, as shown in FIG. 9, the disease-related processing unit 66 has a blood vessel extraction unit 70 that extracts blood vessels, such as a superficial blood vessel, intramucosal bleeding, and extramucosal bleeding, from the special light image, and a determination unit 72 that determines pathological remission or pathological non-remission of ulcerative colitis on the basis of the extracted blood vessel.

The blood vessel extraction unit 70 extracts, as a blood vessel, superficial vascular congestion, intramucosal bleeding, and extramucosal bleeding on the basis of at least one of frequency response or brightness values obtained from the special light image. The determination unit 72 determines pathological remission or pathological non-remission of ulcerative colitis by using an index value that is obtained on the basis of an area of the superficial vascular congestion, an area of the intramucosal bleeding, and an area of the extramucosal bleeding of the special light image. The index value is preferably a value that is obtained by individually adding the areas of the superficial vascular congestion, the areas of the intramucosal bleeding, and the areas of the extramucosal bleeding. Specifically, the determination unit 72 determines that ulcerative colitis is in pathological remission in a case where the index value is less than a threshold value, and that the ulcerative colitis is in pathological non-remission in a case where the index value is a threshold value or more.

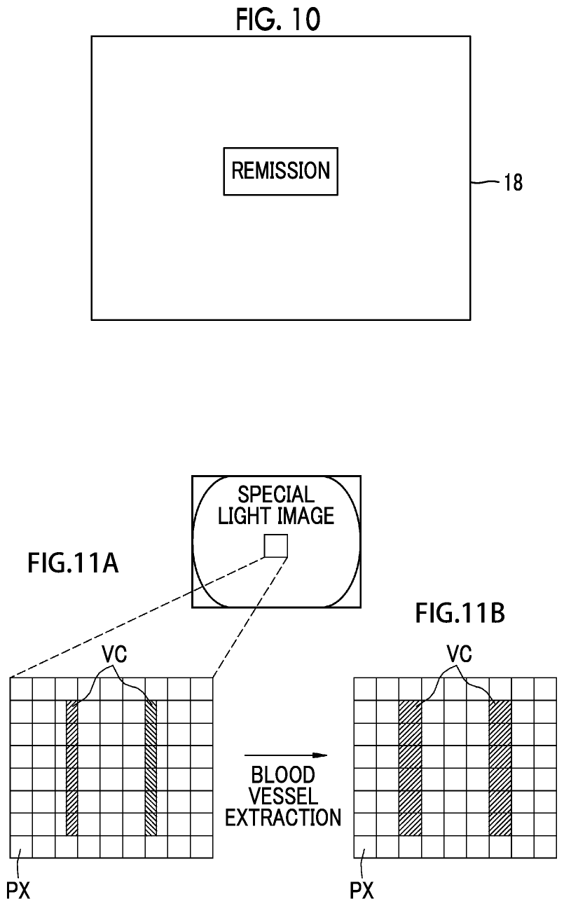

The information regarding the determination by the determination unit 72 is displayed on the monitor 18 and used for the determination of the pathological remission or the pathological non-remission of ulcerative colitis by the user. In a case where the determination unit 72 determines that ulcerative colitis is in pathological remission, a message of the determination result is displayed on the monitor 18, as shown in FIG. 10. In a case where the information regarding the determination is displayed, it is preferable to superimpose and display the special light image that is used for the determination by the determination unit 72.

In order to improve the accuracy of the determination by the determination unit 72, it is preferable to use a special light image in which the observation target is magnified at an appropriate magnification ratio. Specifically, as in the special light of the present embodiment, it is preferable to use a special light image which is obtained by picking up an image of the observation target illuminated with illumination light including short-wavelength narrowband light, the observation target being magnified at a first magnification ratio or more and less than a second magnification ratio, which is more than the first magnification ratio. Here, the narrowband light refers to light having a half-width of 40 nm or less, light emitted as it is from a semiconductor light source such as an LED or LD (for example, “violet light V” of the first embodiment and “violet laser light” and “blue laser light” of the third embodiment), or light in which light from broadband light such as white light is cut out by a filter (for example, “blue narrowband light” and “green narrowband light” of the second embodiment). In a case where blood vessels are extracted by the blood vessel extraction unit 70 for the special light image based on illumination light including short-wavelength narrowband light, the extraction accuracy of a superficial blood vessel, intramucosal bleeding, and extramucosal bleeding extracted by the blood vessel extraction unit 70 is improved as compared with the accuracy of the blood vessel extraction that is performed for an image based on light not including short-wavelength narrowband light. Further, it is preferable that the first magnification ratio is five times or more, and the second magnification ratio is 135 times (in a case where the monitor 18 is 19 inches) to 230 times (in a case where the monitor 18 is 32 inches) or less.

The reason for setting the first magnification ratio to five times is as follows. In a case where far-field imaging or imaging with low magnification ratio in which the thickness of the microvessel is decreased into one pixel or less of the monitor 18 is performed when a microvessel, such as a superficial blood vessel, intramucosal bleeding, and extramucosal bleeding, is extracted, the thickness of the blood vessel is extracted as one pixel even though the actual thickness of the blood vessel is one pixel or less, when the blood vessel is extracted by the blood vessel extraction unit 70. For example, in a case where a vascular density indicating the ratio of the blood vessel to a 72-pixel region which is a specific pixel region is calculated, as shown in (A) of FIG. 11, although the blood vessel VC having a thickness of smaller than one pixel (represented by “PX”, the same applies hereinafter) belongs to actually six pixels and the vascular density is 6/72, the thickness of the blood vessel VC having an actual thickness of less than one pixel is also extracted by the blood vessel extraction unit 70 as one pixel and as shown in (B) of FIG. 11, the blood vessel VC may be extracted as 12 pixels and the vascular density may become 12/72. That is, in a case where the blood vessel VC having a thickness of less than one pixel is extracted as one pixel, a difference from the actual area of the blood vessel occurs. This is one of the causes of lowering the accuracy of the determination result by the determination unit 72.

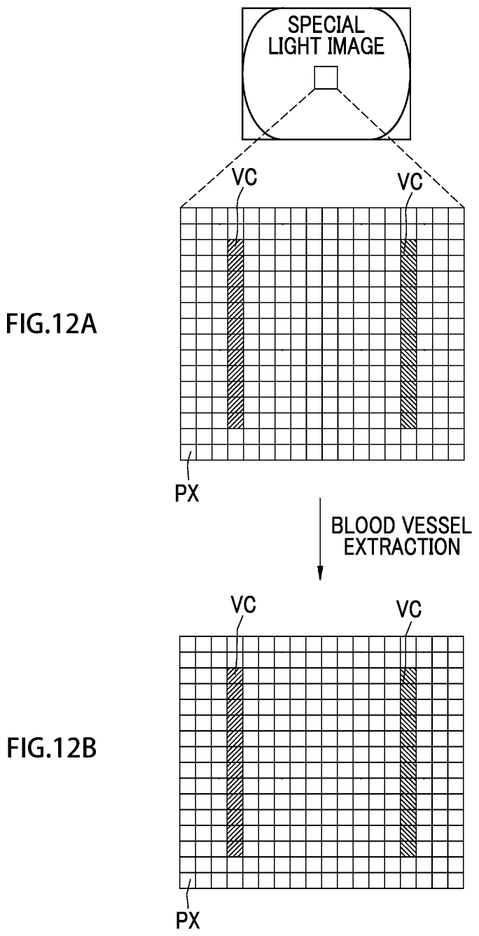

Therefore, it is preferable to magnify the observation target at the first magnification ratio at which the thickness of the blood vessel is made one or more pixels. That is, in a case where a vascular density indicating the ratio of the blood vessel to a 288-pixel region which is a specific pixel region is calculated, and as shown in (A) of FIG. 12, the blood vessel VC belongs to actually 24 pixels and a vascular density is 24/288, the blood vessel is extracted from the special light image in which the observation target is magnified at the first magnification ratio at which the thickness of the blood vessel VC is made one or more pixels, so that as shown in (B) of FIG. 12, the blood vessel VC may be extracted as 24 pixels and the vascular density may become 24/288, like the actual size. As a result, since the area of the extracted blood vessel is the same as the actual area, the accuracy of the determination result by the determination unit 72 can be improved.

In a case where the monitor 18 that displays the special light image is a 19-inch 8K monitor, the size of one pixel is 54 μm. Therefore, in order to put a microvessel having a thickness of 10 μm onto a plurality of pixels, which are one or more pixels, the first magnification ratio is preferably 54 μm/10 μm=5.4 times≈5 times or more.

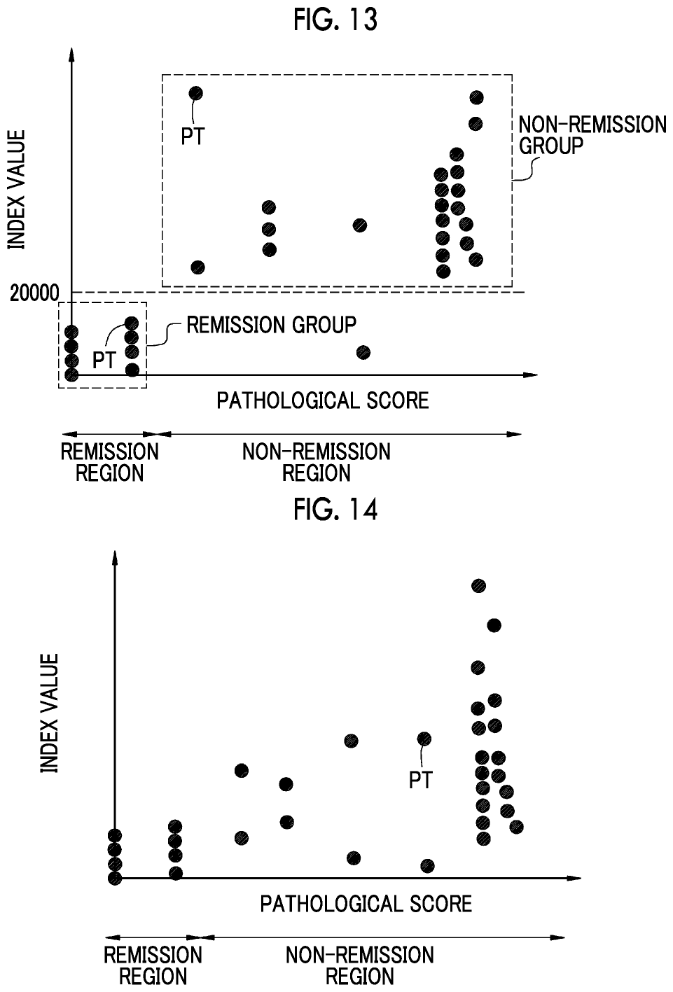

The reason for setting the second magnification ratio to less than 135 times is as follows. In a case where the index value that is obtained on the basis of the area of the superficial vascular congestion, the area of the intramucosal bleeding, and the area of extramucosal bleeding and a pathological score (the larger the score, the higher the severity of the disease) of a disease corresponding to the index value are plotted on a two-dimensional graph, and as shown in FIG. 13, the second magnification ratio is 40 times, which is less than 135 times, points PT's of which the pathological scores are in a pathological remission region are distributed in an index value of 20000 or less. On the other hand, points PT's of which the pathological scores are in a pathological non-remission region are distributed in an index value of 20000 or more. That is, with the index value, a pathological remission group including the points PT's in the pathological remission region and a pathological non-remission group including the points PT's in the pathological non-remission region can be distinguished from each other. Accordingly, the determination by the determination unit 72 can be made.

On the other hand, as shown in FIG. 14, in a case where the second magnification ratio is 135 times, it is difficult to distinguish the points PT's distributed in the pathological remission region and the points PT's distributed in the pathological non-remission region, by using the index value. For this reason, in a case where the second magnification ratio is 135 times, it is difficult to determine the pathological remission or the pathological non-remission of ulcerative colitis by using the index value. Therefore, the second magnification ratio is preferably less than 135 times. Note that, in a case where the monitor 18 is 19 inches, the second magnification ratio is preferably less than 135 times, but in a case where the monitor 18 is 19 inches or more, the second magnification ratio may be 135 times or more. For example, in a case where the monitor 18 is 32 inches, a second magnification ratio in the case of 32 inches into which the second magnification ratio in the case of 19 inches is converted is 230 times or less (135 times (in the case of 19 inches)×39.83 (in the case of 32 inches width)/23.65 (in the case of 19 inches width)).

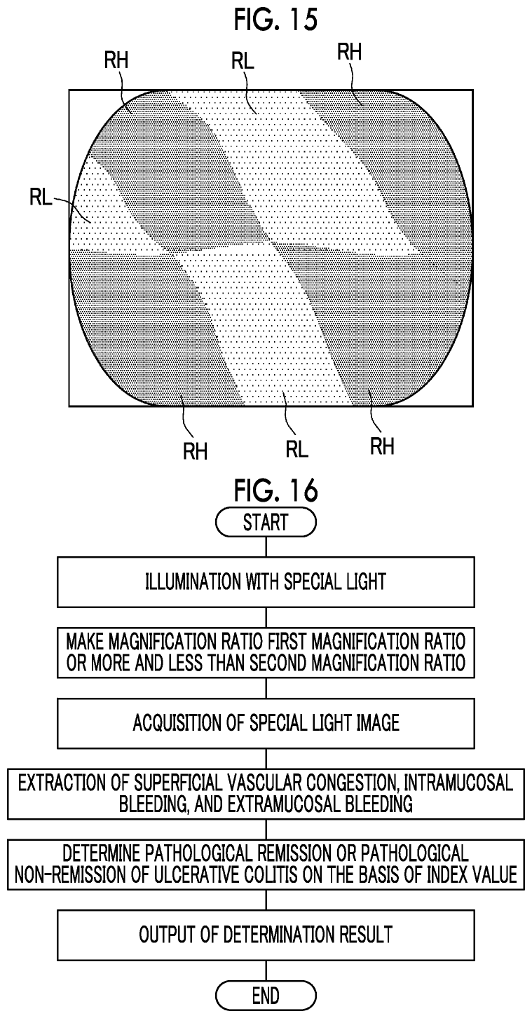

As shown in FIG. 15, in a case where there are blood vessel irregularities in which the vascular density differs depending on the position in the special light image, the areas of the blood vessel extracted in the magnified region are different between a region RH in which the vascular density is locally high and a region RL in which the vascular density is locally low. In this case, in a case where the second magnification ratio exceeds 135 times to 230 times, the magnified image may include only one of the region RL or the region RH, not the average region of the region RL and the region RH. In this case, with the blood vessel extraction, only local blood vessel information can be acquired and average blood vessel information as the entire special light image cannot be obtained. As a result, the accuracy of the determination by the determination unit 72 is decreased.

Next, a series of flow of a disease-related processing mode will be described with reference to a flowchart shown in FIG. 16. In a case where a mode is switched to the disease-related processing mode, the observation target is irradiated with special light including short-wavelength narrowband light. Further, the zoom operation portion 12h is operated, to make the magnification ratio of the observation target the first magnification ratio or more and less than the second magnification ratio. The endoscope 12 picks up an image of the observation target illuminated with special light to obtain a special light image that is one of endoscopic images (medical images). The image acquisition unit 50 acquires the special light image from the endoscope 12.

The blood vessel extraction unit 70 extracts, as a blood vessel, superficial vascular congestion, intramucosal bleeding, and extramucosal bleeding on the basis of frequency response or brightness values obtained from the special light image. The determination unit 72 determines pathological remission or pathological non-remission of ulcerative colitis by using an index value that is obtained on the basis of an area of the superficial vascular congestion, an area of the intramucosal bleeding, and an area of the extramucosal bleeding of the special light image. Information regarding the determination by the determination unit 72 is displayed on the monitor 18.

### Second Embodiment

In the second embodiment, a broadband light source, such as a xenon lamp, and a rotation filter are used to illuminate the observation target, instead of the four-color LEDs 20a to 20e shown in the first embodiment. Further, an image of the observation target is picked up by a monochrome image pickup sensor, instead of the color image pickup sensor 44. Other than that, the same as the first embodiment applies.

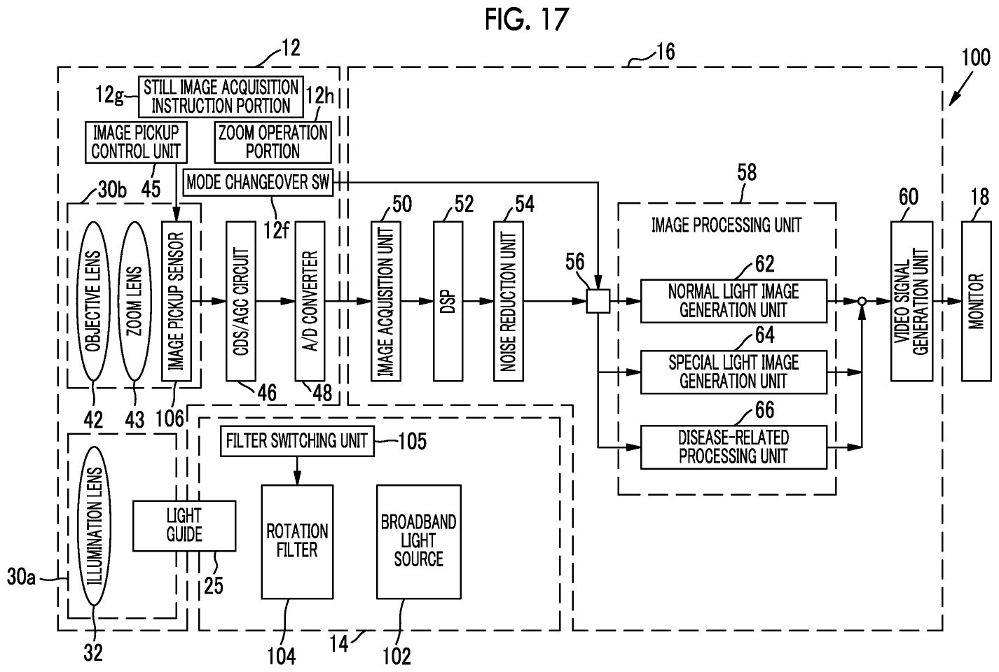

As shown in FIG. 17, in an endoscope system 100 of the second embodiment, the light source apparatus 14 is provided with a broadband light source 102, a rotation filter 104, and a filter switching unit 105, instead of the four-color LEDs 20a to 20e. Further, the image pickup optical system 30b is provided with a monochrome image pickup sensor 106 without a color filter, instead of the color image pickup sensor 44.

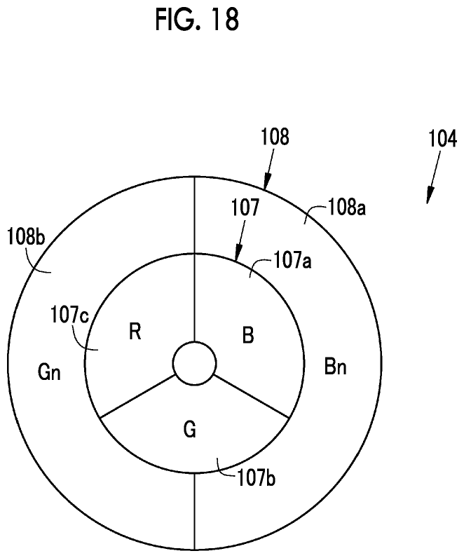

The broadband light source 102 is a xenon lamp, a white LED, or the like, and emits white light having a wavelength range ranging from blue to red. The rotation filter 104 is provided with a filter for normal light mode 107 and a filter for special light mode and disease-related processing mode 108 in order from the inside (see FIG. 18). The filter switching unit 105 moves the rotation filter 104 in a radial direction, and inserts the filter for normal light mode 107 into the optical path of white light in a case where the normal light mode is set by the mode changeover SW 12f, and inserts the filter for special light mode and disease-related processing mode 108 into the optical path of white light in a case where the special light mode or the disease-related processing mode is set.

As shown in FIG. 18, the filter for normal light mode 107 is provided with a B filter 107a that transmits broadband blue light B of white light, a G filter 107b that transmits broadband green light G of white light, and an R filter 107c that transmits broadband red light R of white light, along a circumferential direction. Therefore, in the normal light mode, with the rotation of the rotation filter 104, the observation target is alternately irradiated with the broadband blue light B, the broadband green light G, and the broadband red light R, as normal light.

The filter for special light mode and disease-related processing mode 108 is provided with a Bn filter 108a that transmits blue narrowband light of white light and a Gn filter 108b that transmits green narrowband light of white light, along the circumferential direction. Therefore, in the special light mode or the disease-related processing mode, with the rotation of the rotation filter 104, the observation target is alternately irradiated with the blue narrowband light and the green narrowband light as short-wavelength narrowband light, as special light. The wavelength range of the blue narrowband light is preferably 400 to 450 nm, and the wavelength range of the green narrowband light is preferably 540 to 560 nm.

In the endoscope system 100, in the normal light mode, an image of the observation target is picked up by the monochrome image pickup sensor 106 each time the observation target is illuminated with the broadband blue light B, the broadband green light G, or the broadband red light R. Accordingly, a Bc image signal, a Gc image signal, and an Rc image signal can be obtained. A normal light image is generated by the same method as in the first embodiment, on the basis of the three-color image signals.

In the endoscope system 100, in the special light mode or the disease-related processing mode, an image of the observation target is picked up by the monochrome image pickup sensor 106 each time the observation target is illuminated with the blue narrowband light or the green narrowband light. Accordingly, a Bs image signal and a Gs image signal can be obtained. A special light image is generated by the same method as in the first embodiment, on the basis of the two-color image signals.

### Third Embodiment

In the third embodiment, a laser light source and a phosphor are used to illuminate the observation target, instead of the four-color LEDs 20a to 20e shown in the first embodiment. In the following, only the parts different from the first embodiment will be described, and the description of the parts substantially the same as those of the first embodiment will be omitted.

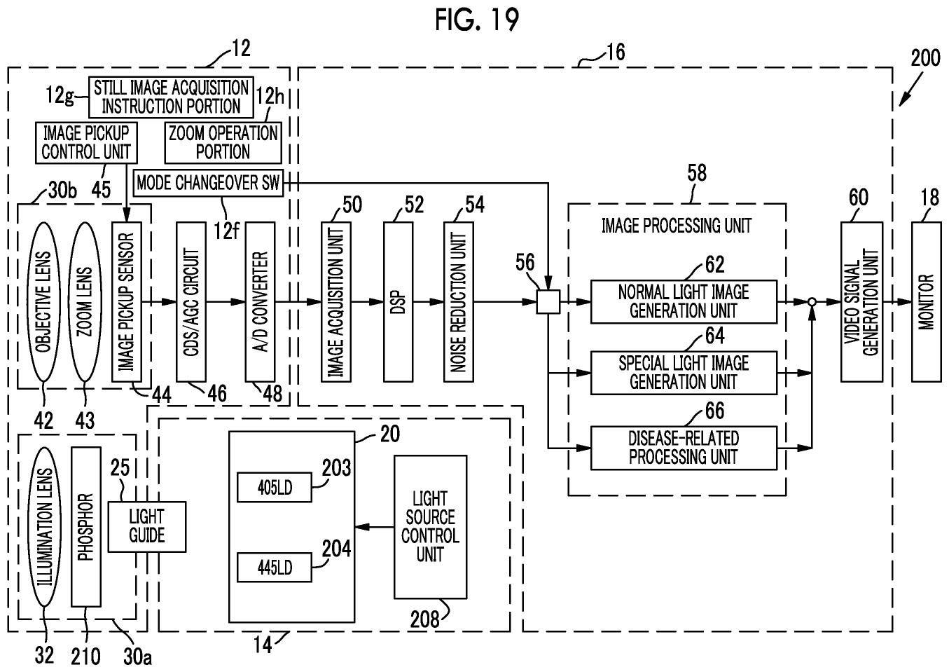

As shown in FIG. 19, in an endoscope system 200 of the third embodiment, the light source unit 20 of the light source apparatus 14 is provided with a violet laser light source unit 203 (denoted by “405LD”, the LD represents a “laser diode”) that emits violet laser light having a central wavelength of 405±10 nm, which corresponds to short-wavelength narrowband light, and a blue laser light source unit 204 (denoted by “445LD”) that emits blue laser light having a central wavelength of 445±10 nm, instead of the four-color LEDs 20a to 20e. The light emitted from semiconductor light emitting elements of the light source units 204 and 206 is individually controlled by the light source control unit 208.

In the normal light mode, the light source control unit 208 makes the blue laser light source unit 204 turned on. On the other hand, in the special light mode or the disease-related processing mode, the light source control unit 208 makes the violet laser light source unit 203 and the blue laser light source unit 204 turned on at the same time.

The half-width of the violet laser light or the blue laser light is preferably about ±10 nm. Further, as the violet laser light source unit 203 or the blue laser light source unit 204, a broad area type InGaN-based laser diode can be used, and an InGaNAs-based laser diode or a GaNAs-based laser diode can also be used. Further, as the light source, a light emitting body such as a light emitting diode may be used.

The illumination optical system 30a is provided with a phosphor 210 to which violet laser light or blue laser light from the light guide 25 is incident, in addition to the illumination lens 32. The phosphor 210 is excited by blue laser light and emits fluorescence. Therefore, the blue laser light corresponds to excitation light. Further, a part of the blue laser light is transmitted without exciting the phosphor 210.

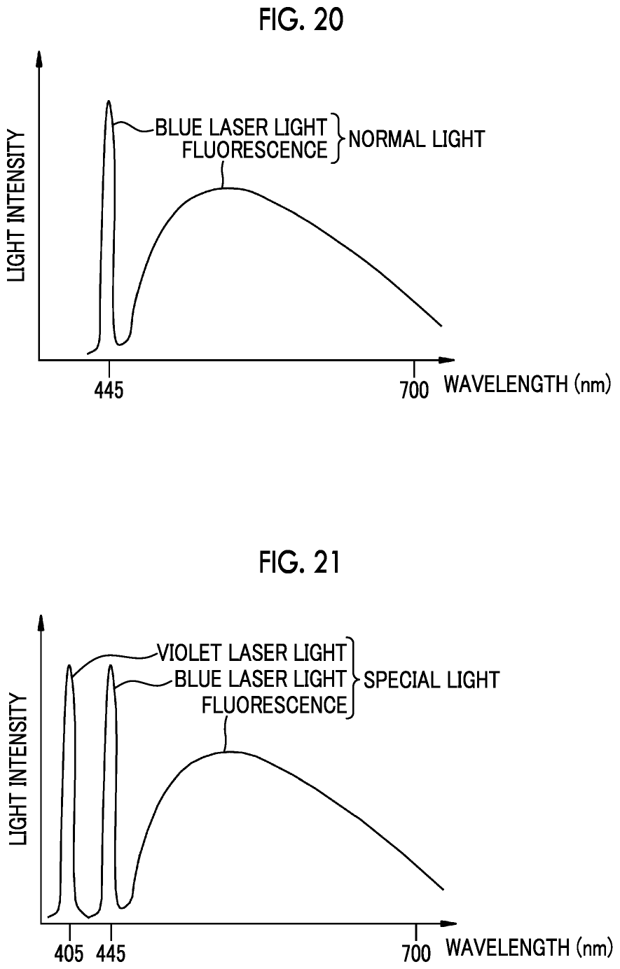

Here, in the normal light mode, since the blue laser light is mainly incident on the phosphor 210, as shown in FIG. 20, the observation target is illuminated with normal light in which the blue laser light and the fluorescence emitted from the phosphor 210 excited by the blue laser light are combined. In a case where an image of the observation target illuminated with the normal light is picked up by the image pickup sensor 44, a normal light image including a Bc image signal, a Gc image signal, and an Rc image signal can be obtained.

Further, in the special light mode or the disease-related processing mode, since violet laser light and blue laser light are simultaneously incident on the phosphor 210, as shown in FIG. 21, pseudo white light including the fluorescence emitted from the phosphor 210 excited by the violet laser light and the blue laser light is emitted, in addition to the violet laser light and the blue laser light, as the special light. In a case where an image of the observation target illuminated with the special light is picked up by the image pickup sensor 44, a special light image including a Bs image signal, a Gs image signal, and an Rs image signal can be obtained. The pseudo white light may be a combination of violet light V, blue light B, green light G, and red light R emitted from V-LED 20a, B-LED 20b, G-LED 20c, and R-LED 20d.

As the phosphor 210, a phosphor that includes a plurality of types of phosphors (for example, a YKG-based phosphor or a phosphor such as BaMgAl₁₀O₁₇ (BAM)) which absorb a part of blue laser light and which excite and emit green color to yellow color is preferably used. As in the present configuration example, in a case where a semiconductor light emitting element is used as an excitation light source for the phosphor 210, high-intensity white light can be obtained with high luminous efficiency, the intensity of white light can be easily adjusted, and the change of the color temperature and chromaticity of white light can be suppressed to a small extent.

In the above-described embodiment, the present invention is applied to an endoscope system that performs processing for an endoscopic image, which is one of medical images, but the present invention can also be applied to a medical image processing system that performs processing for medical images other than the endoscopic image. The present invention can also be applied to a diagnosis support apparatus that is used to provide diagnostic support to a user using a medical image. The present invention can also be applied to a medical service support apparatus that is used to support medical service, such as a diagnostic report, using a medical image.

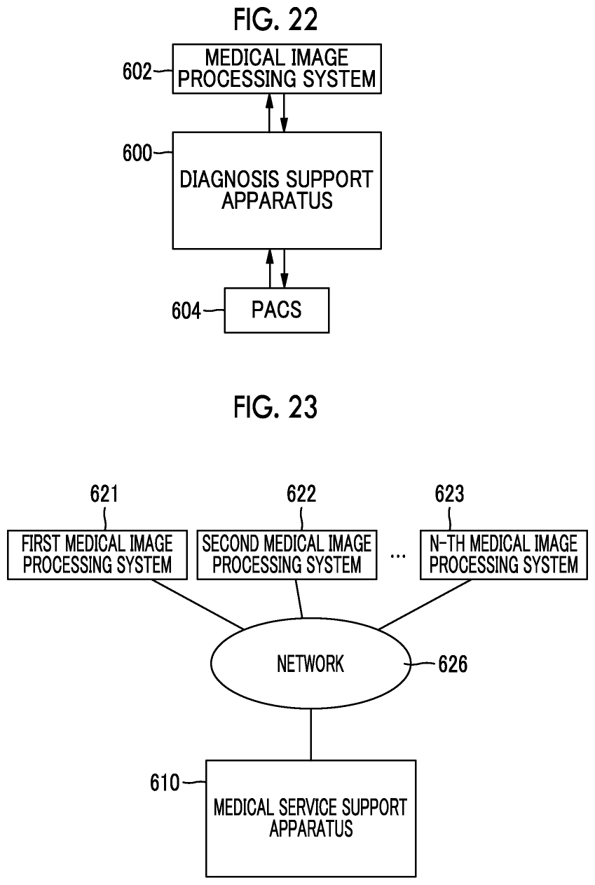

For example, as shown in FIG. 22, a diagnosis support apparatus 600 is used in combination with a modality, such as a medical image processing system 602, and picture archiving and communication systems (PACS) 604. Further, as shown in FIG. 23, a medical service support apparatus 610 is connected to various examination apparatuses, such as a first medical image processing system 621, a second medical image processing system 622, . . . , and an N-th medical image processing system 623, via any network 626. The medical service support apparatus 610 receives medical images from the first to N-th medical image processing systems 621, 622, . . . , to 623, and supports the medical service on the basis of the received medical images.

In the above-described embodiment, as a hardware structure of a processing unit that executes various processing, such as the normal light image generation unit 62, the special light image generation unit 64, the disease-related processing unit 66, the blood vessel extraction unit 70, and the determination unit 72, which are included in the image processing unit 58, various processors as described below are used. The various processors include, for example, a central processing unit (CPU), which is a general-purpose processor that executes software (programs) to function as various processing units, a programmable logic device (PLD), such as a field programmable gate array (FPGA), which is a processor having a changeable circuit configuration after manufacture, and a dedicated electrical circuit, which is a processor having a dedicated circuit configuration designed to execute various processing.

One processing unit may be constituted of one of the various processors or may be constituted of a combination of two or more processors of the same type or different types (for example, a combination of a plurality of FPGAs and a combination of a CPU and an FPGA). Further, the plurality of processing units may constitute one processor. A first example of the configuration in which the plurality of processing units are constituted of one processor is an aspect in which one or more CPUs and software are combined to constitute one processor and the processor functions as a plurality of processing units. A representative example of the aspect is a computer such as a client or server. A second example of the configuration is an aspect in which a processor that implements all of the functions of a system including the plurality of processing units with one integrated circuit (IC) chip is used. A representative example of the aspect is a system on chip (SoC). As described above, as the hardware structure of various processing units, one or more of the various processors are used.

Furthermore, as the hardware structure of the various processors, more specifically, an electrical circuit (circuitry) in which circuit elements, such as semiconductor elements, are combined are used.

### EXPLANATION OF REFERENCES

10: endoscope system

12: endoscope

12a: insertion part

12b: operation part

12c: bendable part

12d: distal end part

12e: angle knob

12f: mode changeover switch

12g: still image acquisition instruction portion

12h: zoom operation portion

14: light source apparatus

16: processor apparatus

18: monitor

19: user interface

20: light source unit

20a: V-LED

20b: B-LED

20c: G-LED

20d: R-LED

21: light source control unit

23: optical path coupling unit

25: light guide

30a: Illumination optical system

30b: image pickup optical system

32: illumination lens

42: objective lens

43: zoom lens

44: image pickup sensor

45: image pickup control unit

46: CDS/AGC circuit

47: magnification ratio display section

48: A/D converter

49: magnification ratio display section

49a: horizontal bar

49b: upper limit display bar

50: image acquisition unit

52: DSP

54: noise reduction unit

56: image processing switching unit

58: image processing unit

60: video signal generation unit

62: normal light image generation unit

64: special light image generation unit

66: disease-related processing unit

70: blood vessel extraction unit

72: determination unit

100: endoscope system

102: broadband light source

104: rotation filter

105: filter switching unit

106: image pickup sensor

107: filter for normal light mode

107a: B filter

107b: G filter

107c: R filter

108: filter for special light mode and disease-related processing mode

108a: Bn filter

108b: Gn filter

200: endoscope system

203: violet laser light source unit

204: blue laser light source unit

208: light source control unit

210: phosphor

600: diagnosis support apparatus

602: medical image processing system

604: PACS

610: medical service support apparatus

621: first medical image processing system

622: second medical image processing system

623: N-th medical image processing system

626: network

Bx1, Bx2, Bx3, Bx4: box

V: violet light

B: blue light

G: green light

R: red light

SC: specific color

VC: blood vessel

PX: pixel

PT: point

RH, RL: region

What is claimed is:

1. An image processing apparatus comprising:
a processor configured to:
acquire a medical image which is obtained by picking up an image of an observation target illuminated with illumination light including short-wavelength narrowband light, the observation target being magnified at a first magnification ratio or more and less than a second magnification ratio that is more than the first magnification ratio; and
perform processing related to a disease on the basis of the medical image.

2. The image processing apparatus according to claim 1,
wherein magnification of the observation target is performed at the first magnification ratio, to make a thickness of a blood vessel that is included in the observation target magnified to one or more pixels.

3. The image processing apparatus according to claim 1,
wherein the first magnification ratio is five times or more.

4. The image processing apparatus according to claim 1,
wherein the second magnification ratio is 230 times or less.

5. The image processing apparatus according to claim 1,
wherein the illumination light is violet light of which a central wavelength or a peak wavelength includes a wavelength of 410 nm, as the short-wavelength narrowband light.

6. The image processing apparatus according to claim 1,
wherein the illumination light is blue narrowband light and green narrowband light, as the short-wavelength narrowband light, and
the medical image is obtained by picking up an image of the observation target that is alternately illuminated with the blue narrowband light and the green narrowband light.

7. The image processing apparatus according to claim 1,
wherein the illumination light is pseudo white light including the short-wavelength narrowband light and fluorescence obtained by irradiating a phosphor with excitation light.

8. The image processing apparatus according to claim 1,
wherein the illumination light includes violet light as the short-wavelength narrowband light and blue light, green light, or red light.

9. The image processing apparatus according to claim 1,
wherein the processor further configured to perform at least one of calculating an index value for a stage of ulcerative colitis, determining the stage of the ulcerative colitis, or determining pathological remission or pathological non-remission of the ulcerative colitis, on the basis of at least one of superficial vascular congestion, intramucosal bleeding, or extramucosal bleeding that is obtained from the medical image.

10. The image processing apparatus according to claim 1,
wherein the processor further configured to calculate an index value related to a disease on the basis of the medical image, and
the index value distinguishes between pathological remission and pathological non-remission of a disease in a two-dimensional graph showing the correspondence between the index value and a severity of the disease corresponding to the index value.

11. An endoscope system comprising:
a light source unit that emits illumination light including short-wavelength narrowband light; and
a processor configured to:
acquire a medical image which is obtained by picking up an image of an observation target illuminated with illumination light including short-wavelength narrowband light, the observation target being magnified at a first magnification ratio or more and less than a second magnification ratio that is more than the first magnification ratio; and
perform processing related to a disease on the basis of the medical image.

12. An operation method of an image processing apparatus, the method comprising:
acquiring a medical image which is obtained by picking up an image of an observation target illuminated with illumination light including short-wavelength narrowband light, the observation target being magnified at a first magnification ratio or more and less than a second magnification ratio that is more than the first magnification ratio; and
performing processing related to a disease on the basis of the medical image.
